(19) United States
(12) Patent Application Publication
(10) Pub. No.: US 2023/0200866 A1
(43) Pub. Date: Jun. 29, 2023

(54) IMPROVED LINEAR FIBULAR NAIL

(71) Applicant: MEDARTIS AG, Basel (CH)

(72) Inventor: Darryl AUSTON, Westminster, CO (US)

(73) Assignee: MEDARTIS AG, Basel (CH)

(21) Appl. No.: 17/996,308

(22) PCT Filed: Apr. 15, 2021
(86) PCT No.: PCT/US2021/027464

§ 371 Date: Oct. 14, 2022

Related U.S. Application Data
(60) Provisional application No. 63/010,340, filed on Apr. 15, 2020.

(51) Int. Cl. A61B 17/72; A61B 17/68

(52) U.S. Cl. CPC A61B 17/7291; A61B 17/7283; A61B 17/725; A61B 17/683; A61B 2017/0404

(57) ABSTRACT

A fibular nail for repairing distal fibular fractures is provided that does not require a transfibular-transtibial interlocking screw for stabilization. The nail provides transverse apertures permitting fixation of the proximal and distal portions of a fractured fibula to the nail. The nail has apertures for securing the fibula to the nail on the proximal side of a fracture and on the distal side of a fracture. Optionally, the nail may be connected to the tibia for stabilization.

### CROSS-REFERENCE TO RELATED APPLICATIONS

This application claims the benefit of U.S. Provisional Patent Application No. 63/010,340, filed on Apr. 15, 2020, the entire content of which is hereby incorporated by reference.

### BACKGROUND OF THE INVENTION

Fibula break. Often, the break is near the distal end of the fibula. The fibula is connected to the tibia at the distal end by ligaments, such as the interosseous ligament, transverse tibiofibular ligament, anterior inferior tibiofibular ligament and the posterior inferior tibiofibular ligament. The resulting syndesmosis creates a strong, but indirect, connection between the weight bearing tibia and the fibula, which serves as an attachment point for various muscle groups and carries little weight. The syndesmodic connection is not rigid and permits small movements between the fibula and tibia, which increases the range of motion of the ankle. Fibula breaks often do not irreparably damage the syndesmosis.

Fibular breaks are often repaired by open reduction and internal fixation. Such requires surgical exposure of the fibula sufficient to permit direct reduction of the fracture fragments. The exposure must extend along the bone a sufficient length to accommodate plate fixation. The resulting wound may be extensive and often proves troublesome, especially for geriatric or diabetic patients.

Because fibular breaks may displace the fibula from the tibia, surgical repair often includes mechanical fixation of the fibula to the tibia with a transfibular-transtibial interlocking screw. Alternatively, Smith & Nephew developed a method of attaching the fibula to the tibia with a flexible suture it calls the “ULTRATAPE” suture. The ULTRATAPE suture is part of the INVISIKNOT system that permits micro-motion between the tibia and fibula, mimicking syndesmosis. The INVISIKNOT system may be used with plates used to repair fibula fractures.

An alternative known repair method uses the insertion of a fibular nail in the fibular canal. Fibular nails stabilize the fractured fibula and may be preferable for certain fracture patterns and for certain patient populations. Fibular nail insertion creates less soft tissue trauma than open reduction and internal fixation techniques. A prior art fibular nail sold by Acumed is depicted in FIG. 1. As can be seen, the nail has an enlarged distal end (distal from the patient's perspective) with two anterior to posterior distal interlocking screws and a transfibular-transtibial interlocking screw.

In many instances, the break in the fibula will approximate a plane cutting diagonally across the axis of the fibula. The proximate and distal portions of the broken fibula will often slide in opposite directions across the diagonal break with the resulting length of the broken fibula being less that the unbroken bone. To repair the break, the fibula must be realigned and returned to its original length. The fibular nail is inserted and is attached to the distal portion of the broken fibula with the anterior to posterior distal interlocking screws. In one prior art system, transfibular-transtibial interlocking screw(s) are inserted to rigidly fix the fibular nail to the tibia. The transfibular-transtibial interlocking screw(s) assure rotational fixation of the nail with the fibula and prevent axial compression of the fibula. In another prior art system, talons are deployed in the proximate end of the nail to engage the fibula medullary wall and axially and rotationally fix the nail to the fibula.

Prior art fibular nails are bent. As shown in FIG. 1, the axis of the distal enlarged portion of the nail is canted relative to the axis of the narrower proximate portion of the nail, when viewed from the anterior or posterior. This bend limits the length of the enlarged portion of the nail that is available for anterior to posterior interlocking screw placement. Typically, the enlarged portion of the nail extends approximately four centimeters before tapering down to the narrower proximate end of the nail. The bend also limits both the direction and location of the transfibular-transtibial interlocking screw(s). Additionally, the prior art fibular nails typically require rigid fixation of the fibula to the tibia, which limits the range of motion possible upon recovery.

What is needed is a simple fibular nail that does not require transfibular-transtibial interlocking screw(s) to rotationally and transitionally fix the repaired fibula.

### SUMMARY OF THE INVENTION

The present invention facilitates repairing a distal fibular fracture with a nail that does not require rigid fixation of the repaired fibula to the tibia. If the distal end of the fibula has separated from the tibia, aspects of the present invention permit non-rigid securement of the fibula to the tibia that mimics syndesmosis.

### BRIEF DESCRIPTION OF THE DRAWINGS

FIG. 1 depicts a prior art fibular nail and associated fibula and tibia.

FIG. 2 depicts an anterior view of an embodiment of the present invention.

FIG. 3 depicts a lateral to medial view of an embodiment of the present invention.

### DETAILED DESCRIPTION

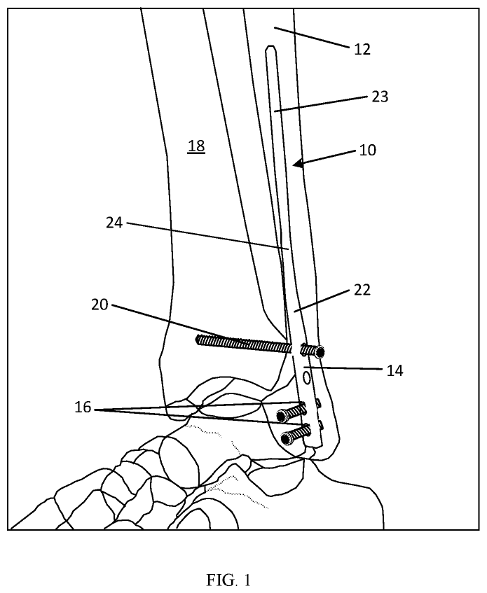

FIG. 1 depicts a prior art fibular nail 10 made by Acumed. FIG. 1 is a schematic representation of how the nail would be positioned in a patient. The prior art fibular nail 10 is inserted in the fibula 12. At its distal end, the prior art fibular nail 10 has an enlarged portion 14 with apertures receiving anterior to posterior distal interlock screws 16. The tibia 18 is connected to the prior art fibular nail 10 by a transfibular-transtibial interlocking screw 20. The prior art fibular nail 10 has a taper 22 that transitions the prior art fibular nail 10 from the enlarged portion 14 to the proximate end 23 of the prior art fibular nail 10. As can be seen, the prior art fibular nail 10 has a bend 24 such that the proximate end 23 of the prior art fibular nail 10 is canted relative to the enlarged portion 14 of the prior art fibular nail 10 when viewed from the anterior or posterior.

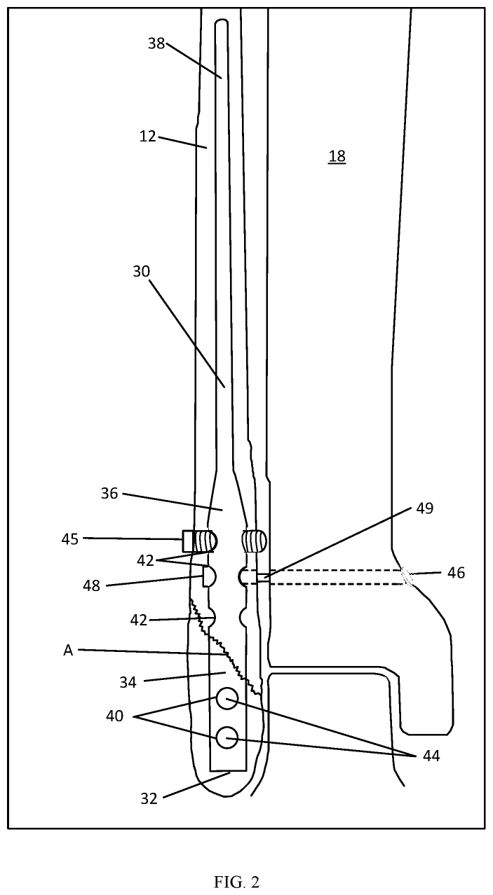

FIG. 2 depicts an anterior view of an embodiment of the present invention. The linear fibular nail 30 is shown in a fibula 12 having a fracture line A that is representative of a typical fibular fracture. Beginning at the distal end 32 of the linear fibular nail 30 is an extended enlarged portion 34 with a taper 36 at the proximate end of the extended enlarged portion 34. The linear fibular nail 30 is solid but one of skill would recognize that, with proper material selection, the nail could be hollow. The taper 36 transitions the linear fibular nail 30 extended enlarged portion 34 down to the proximate portion 38 of the linear fibular nail 30, which has a smaller diameter than the extended enlarged portion 34. The extended enlarged portion 34, in one embodiment, extends approximately six centimeters from the distal end 32 of the linear fibular nail 30 before taper 36 begins. Those of skill will recognize that the available space within the distal end of a fibula 12 varies from patient to patient. Six centimeters provides sufficient length to practice the invention on the majority of patients. Those of skill will also recognize that nail shapes other than the gradual taper 36 depicted could be used to effectively increase the length of the extended enlarged portion 34 beyond what is depicted in the embodiment shown.

The extended enlarged portion 34 of the linear fibular nail 30 has two anterior to posterior interlocking screw apertures 40 near the distal end 32. Proximate the anterior to posterior locking screw apertures 40 on the extended enlarged portion 34 are three lateral to medial locking screw apertures 42. One of skill would recognize that in another embodiment, the axial orientation of the anterior to posterior interlocking screw apertures 40 and of the lateral to medial locking screw apertures 42 could be varied. In other words, in other embodiments, the orientations of the screws need not be precisely anterior to posterior or lateral to medial.

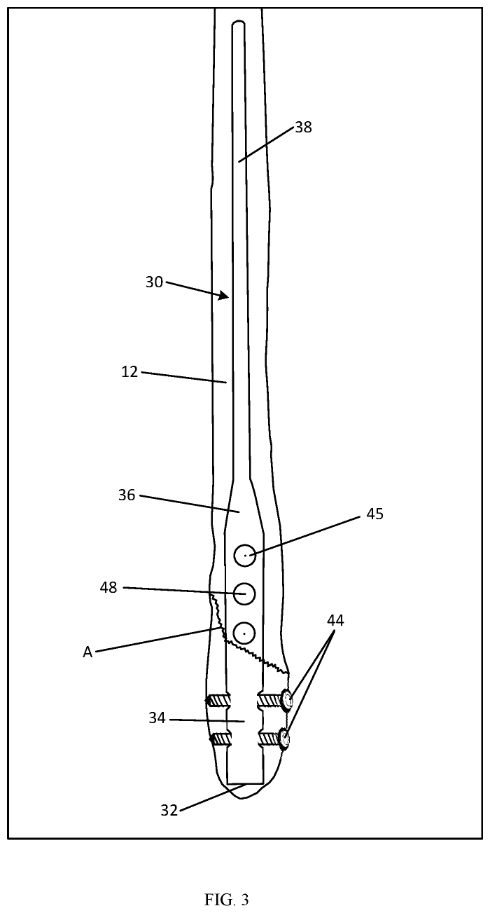

In practice, the broken fibula 12 is realigned to approximate its original length. A clamp (not shown) mediated reduction may be required to properly position the broken fibula 12. Once the broken fibula is properly repositioned, the linear fibular nail 30 may be inserted into the fibula 12 medullary cavity. Anterior to posterior interlocking screws 44 are inserted into the anterior to posterior locking screw apertures 40 and secure the distal end of the broken fibula 12 to the linear fibular nail 30. As shown, the anterior to posterior interlocking screws 44 are distal of the fracture line A, as more clearly shown in FIG. 3, a lateral to medial view of the linear fibular nail 30. A lateral to medial interlocking screw 45 attaches the proximal portion of the broken fibula 12 to the linear fibular nail 30, which rotationally and longitudinally fixes the distal and proximal portions of the broken fibula 12 relative to each other.

In an alternate embodiment (not shown), the anterior to posterior interlocking screws 44 and the lateral to medial interlocking screw 45 threadingly engage the linear fibular nail 30. The threading engagement facilitates securing the linear fibular nail 30 to one side of the cortical fibular bone, which adequately stabilizes the broken fibula 12 without creating trauma to the cortical bone on the remote (from the perspective of the surgeon) side of the linear fibular nail 30. In another embodiment, the lengths of the anterior to posterior interlocking screws 44 and the lateral to medial interlocking screw 45 are selected to be slightly less than the diameter of the fibula 12 at the point of attachment. When used, these interlocking screws do not protrude beyond the remote periphery of the broken fibula 12, which safeguards soft tissue on the remote side of the attachment point. Using either of these attachment methods avoids trauma to soft tissue on the remote side of the connection point caused by drilling or by a protruding screw.

Unlike the prior art nail, if the syndesmotic ligaments are not damaged, there is no need to cause trauma to the tibia 18 by boring into the tibia 18. Unlike the prior art, a rigid connection between the tibia 18 and fibula 12 is not required to rotationally or longitudinally fix the proximal portion of a fractured fibula 12 to the distal portion of the fractured fibula 12. Advantageously, the flexibility inherent with syndesmosis is not degraded.

Optionally, if the fibula 12 has displaced from the tibia 18, a non-rigid connector, such as the Smith & Nephew INVISIKNOT system can secure the linear fibular nail 30 to the tibia 18. As shown, the INVISIKNOT medial button 46 is shown on the tibia 18 and the lateral button 48 is adjacent one of the lateral to medial locking screw apertures 42. The INVISIKNOT suture 49 can be seen in the space between the fibula 12 and tibia 18. The suture 49 maintains tension between the lateral button 48 and the medial button 46 that keeps the fibula 12 properly positioned relative to the tibia 18. If rigid fixation between the fibula 12 and tibia 18 is desired, one of the lateral to medial locking screw apertures 42 can be used to engage a transfibular-transtibial interlocking screw 20, as shown in FIG. 1.

As shown, the extended enlarged portion 34 provides additional space for lateral to medial screws 45 that can be used to secure the proximate portion of a broken fibula 12. The extended enlarged portion 34 is possible because the linear fibular nail 30 is not bent like prior art nails. While the extended enlarged portion 34 is coaxial with the proximate portion 38 of the disclosed linear fibular nail 30, those of skill in the art will recognize that nails having additional space for proximal fixation need not have perfectly coaxial distal and proximate portions. Those of skill will recognize that the ability to fix the proximal and distal portions of a broken fibula 12 according to the present invention may be achieved with a fibular nail having a variety of shapes and the invention is not intended to be limited to the embodiment disclosed. For example, a fibular nail with a non-circular cross-section is intended to fall within the claims. Additionally, one of skill would recognize that a nail with a slight bend could be used to practice the claimed invention if the bend allowed insertion of the nail into the fibula such that apertures in the nail are adjacent stable bone on the distal and proximal side of the fibular fracture.

What is claimed is:

1. A fibular nail comprising:
a distal end;
an enlarged portion;
a proximal portion;
a transitional portion, said enlarged portion extending from said distal end to said transitional portion, said enlarged portion having a first cross-section, said transitional portion transitioning from said enlarged portion to said proximal portion, said proximal portion having a second cross-section, said second cross-section having an area less than said first cross-section;
a first aperture extending through said fibular nail in a distal portion of said enlarged portion, said first aperture adapted to receive a first connector, said first connector adapted to fix said fibular nail to a distal portion of a fibula; and
a second aperture extending through said enlarged portion proximal said first aperture, said second aperture adapted to receive a second connector, said second connector adapted to fix said fibular nail to a proximal portion of said fibula when said proximal portion of said fibula is separated from said distal portion of said fibula by a fracture.

2. The fibular nail of claim 1 wherein said proximal portion of said fibular nail is coaxial with said enlarged portion of said fibular nail.

3. The fibular nail of claim 1 wherein said enlarged portion has a third aperture extend through said enlarged portion, said third aperture adapted to receive a third connector, said third connector adapted to connect said fibular nail to a patient's tibia.

4. The fibular nail of claim 1 wherein said enlarged portion is greater than 5 centimeters long.

5. The fibular nail of claim 1 wherein said enlarged portion is approximately 6 centimeters long.

6. The fibular nail of claim 1 further comprising:
a third aperture extending through said enlarged portion, said third aperture adapted to receive a third connector, said third connector adapted to fix said fibular nail to said distal portion of a fibula, said first and third apertures adapted to be oriented anterior to posterior when implanted; and
a fourth aperture extending through said enlarged portion, said fourth aperture adapted to receive a fourth connector, said second and said fourth apertures adapted to be oriented lateral to medial when implanted.

7. The fibular nail of claim 6 wherein said fourth aperture is adapted to receive a fourth connector, said fourth connector adapted to connect said fibular nail to a patient's tibia.

8. The fibular nail of claim 6 wherein said first and third connectors are screws.

9. The fibular nail of claim 1 wherein said first and said second cross-section are circles.

10. The fibular nail of claim 6 wherein said fourth connector comprises a suture.

11. The fibular nail of claim 1 wherein one of said first and said second apertures are threaded.

12. A fibular nail system comprising:
a fibular nail, said fibular nail having a proximal end and a distal end;
a distal fixation screw;
a proximal fixation screw;
a first aperture through said fibular nail;
a second aperture through said fibular nail;
said first aperture adapted to receive said distal fixation screw;
said second aperture adapted to receive said proximal fixation screw; and
said first aperture and said second aperture spaced such that said first aperture is adapted to be adjacent a fibula distal of a fracture and said second aperture adapted to be adjacent said fibula proximal of said fracture.

13. A method of stabilizing a fibula having a fracture at its distal end comprising the steps of:
inserting a fibular nail into the intramedullary fibular canal, said fibular nail having a first and a second transverse aperture;
positioning said fibular nail in said fibular canal such that said first transverse aperture is distal of said fracture and said second transverse aperture is proximal of said fracture;
inserting a first connector into said first transverse aperture, said first connector rotationally and longitudinally fixing said fibular nail to a first portion of said fibula distal of said fracture; and
inserting a second connector into said second transverse aperture, said second connector rotationally and longitudinally fixing said fibular nail to a second portion of said fibula proximal of said fracture.

14. The method of claim 11 wherein said first and said second connectors are screwed into said fibula.

15. The method of claim 11 additionally comprising the step of connecting said fibular nail to a tibia by inserting a third connector into a third transverse aperture in said fibular nail and said third connector engaging said tibia.

16. The method of claim 13 wherein said third connector is a transfibular-transtibial interlocking screw.

17. The method of claim 13 wherein said third connector prevents movement of said fibula only along the axis of said third connector in a direction away from said tibia.

18. The method of claim 11 wherein said first connector is oriented in the anterior to posterior direction.

19. The method of claim 16 wherein said second connector is oriented in the lateral to medial direction.

20. The method of claim 16 wherein said fibular nail has an enlarged distal portion through which said first, second and third transverse apertures are formed and a proximal portion, said enlarged distal portion having a first diameter; said proximal portion having a second diameter and said first diameter is larger than said second diameter and said enlarged distal portion and said proximal portion being coaxial.

21. The method of claim 13 wherein one of said first or said second transverse apertures is threaded.
